(19) United States
(12) Patent Application Publication
(10) Pub. No.: US 2008/0045799 A1
(43) Pub. Date: Feb. 21, 2008

(54) Multipurpose diseased tissue detection devices, systems, and methods

(76) Inventors: Peter Whitehead, West Vancouver (CA); Terence Gilhuly, Vancouver (CA); Pierre Lane, Vancouver (CA)

Correspondence Address: GRAYBEAL, JACKSON, HALEY LLP, 155 - 108TH AVENUE NE, SUITE 350, BELLEVUE, WA, 98004-5973, US

(21) Appl. No.: 11/786,462

(22) Filed: Apr. 10, 2007

Related U.S. Application Data
(60) Provisional application No. 60/790,995, filed on Apr. 10, 2006.

(51) Int. Cl. A61B 1/06

(52) U.S. Cl. 600/178

(57) ABSTRACT

An autofluorescence light examination system having a plurality of modular parts includes a main body, a light source, and at least two detachable instruments. The main body is configured to deliver light to a target tissue and contains at least one optical element configured so that a user can directly view the target tissue through the main body while the target tissue is illuminated by the light source. The main body is further configured to accept at least two detachable instruments that are configured to deliver light to at least two different target tissues located at at least two different areas of the body and configured such that a user can directly view the target tissue through the main body while the target tissue is illuminated by the light source.

### CROSS-REFERENCE TO RELATED APPLICATIONS

The present application claims priority from U.S. provisional patent application Ser. No. 60/790,995, filed Apr. 10, 2006, which is incorporated herein by reference in its entirety and for all its teachings and disclosures.

### BACKGROUND

Autofluorescence is useful for finding diseases such as precancerous and cancerous lesions. Autofluorescence detection can be used to reveal different types of cancers, including skin, cervical, oral and other. Examples of systems that use autofluorescence for disease detection can be found in U.S. patent publication no. 20050234526 and U.S. patent publication no. 20060241347, incorporated herein by reference in their entirety and for all their teachings and disclosures.

One aspect of the detection of autofluorescence is the excitation light source for evoking the autofluorescence, and filtering to remove reflected excitation light, unwanted external light and to increase the signal to noise (background) ratio for light in the spectral region of interest. The interface to and ergonomics of the light source device—how it is held and used by a practitioner—can be made common to multiple direct viewing applications. For the practitioner to have different devices each specialized for each of many different diseases (for example, one device each for detection of oral, skin, cervical and gastrointestinal diseases), would be expensive and could reduce the likelihood of the practitioner more thoroughly investigating multiple diseases.

Accordingly, there has gone unmet a need for a single device or system with a common core and specialized attachments to suit the varied environments and needs for the individual diseases. The present invention provides these and/or other advantages.

### SUMMARY

In one aspect, the current devices, systems, methods, etc., comprise a multipurpose detector of diseases such as pre-malignant and malignant tissues, using direct viewing of the tissues' autofluorescence to indicate disease state. For example, the multiple purposes can concern multiple tissue types, and targeted tissues can include, for example, oral, intestinal, cervical and epidermal. The systems can also, in some configurations, provide for examination of other diseases and use other light sources and examination modalities.

In order to access the several different tissue types, the current devices, systems, methods, etc., comprise modularity. Different viewing and light receiving elements can be contained so as to provide for improved viewing of the particular tissue.

Thus, in one aspect the current devices, systems, methods, etc., are directed to examination systems such as autofluorescence light examination systems, comprising a plurality of modular parts including a) a main body, typically a hand held unit, configured to deliver light to a target tissue and containing at least one optical element configured so that a user can directly view the target tissue through the main body while the target tissue is illuminated by the light source, the main body further configured to accept at least two detachable instruments that are differently configured to deliver light to at least two different target tissues located at at least two different areas of the body (e.g., one for the nose, one for the mouth, one for an ear, one for the vagina, one for the colon), and configured such that a user can directly view the target tissue through the main body while the target tissue is illuminated by the light source, b) the light source, and c) the at least two detachable instruments.

In some embodiments, the system can further comprise at least 3 or 4 or more detachable instruments configured to deliver light to at least three, four or more different target tissues located at at least three, four or more different areas of the body. The system further can comprise at least two detachable viewing eyepieces configured for the delivery of examination light to the user while the user directly views the target tissue through the main body while the target tissue can be illuminated by the light source. The at least two detachable viewing eyepieces can be configured to deliver light from at least two different target tissues located at at least two different areas of the body. The system can further comprise a disposable window element sized to cover a distal end of the detachable instrument and to protect the patient and user from spread of contagion.

In another aspect the current devices, systems, methods, etc., are directed to methods of light examination, typically including autofluorescent light examination of a target tissue comprising using a system or kit as described herein to directly examine with autofluorescent light multiple different target tissues located at multiple different areas of a body without changing the main body.

In some embodiments, the methods can further comprise using 3 or 4 or more detachable instruments configured to deliver light to at least three, four or more different target tissues located at at least three, four or more different areas of the body. The methods can further comprise changing or not changing the light source and/or changing between at least two detachable viewing eyepieces configured for the direct delivery of examination light to the user while the user directly views the target tissue through the main body while the target tissue can be illuminated by the light source. The methods can also comprise changing between at least two same or different disposable window elements sized to cover a distal end of one ore more of the detachable instruments. The methods can additionally comprise obtaining a digital or photographic image of the target tissues under examination and/or obtaining at least one biopsy of at least one of the target tissues under examination.

One advantage of the present current devices, systems, methods, etc., is to provide a cost effective approach of permitting a health practitioner to use autofluorescence, etc., in their practice. Initial costs are difficult to quickly recoup through current billing practices; the current devices, systems, methods, etc., would allow the fixed equipment costs to be divided amongst the different indications.

Turning next to a more general discussion of the current systems, methods, etc., the systems relate to direct viewing of autofluorescence (i.e., an investigator such as a nurse or doctor looking directly at a target area under examination; this may or may not be accomplished with a concomitant imaging device(s), such as digital or photographic imaging). Reports indicate that such direct viewing for the detection of malignant tissues can offer some substantial advantages. For example, fluorescence bronchoscopy as an adjunct to white-light bronchoscopy has been shown to increase the detection of preinvasive cancers. In a multicenter trial conducted by Lam and colleagues [Lam 1998] using a commercial device for clinical use (LIFE-Lung, Xillix Technologies), sensitivity for preinvasive or invasive cancer increased from 25% to 67% with the addition of fluorescence bronchoscopy (relative sensitivity 2.7). See, e.g., Lam, S; Kennedy, T; Unger, M; Miller, Y E; Gelmont, D; Rusch, V; Gipe, B; Howard, D; LeRiche, J C; Coldman, A; Gazdar, A F. Localization of Bronchial Intraepithelial Neoplastic Lesions by Fluorescence Bronchoscopy. Chest 1998; 113(3):696-702. Lam, S. Early Bronchoscopic Diagnosis of Lung Cancer. 10th World Congress of Bronchology and Bronchoesophagology, Budapest, Hungary. 1998 (Abstract) Lam, S; MacAulay, C. Endoscopic Localization of Preneoplastic Lung Lesions. Clinical and Biological Basis of Lung Cancer Prevention, Ed. Martinet, Y; Hirsch, F R; Vignaud, J-M; Mulshine, J L. 1998. Lam, S; Palcic, B. New Bronchoscopic Approaches for the Detection of Early Lung Cancer. Primary Care and Cancer 1998 (May); 18: 17-21.

As another reported example, direct visualization of autofluorescence has also been used for margin delineation of skin cancer. In a study of patients with basal cell carcinoma (BCC) conducted by Lui and colleagues [2001], tumor margins were delineated under white light and then using direct fluorescence visualization. Of the margins tested, fluorescence visualization more accurately estimated the histological margins of the BCC as compared to standard white light examination. As a further reported example, whole-field imaging of the cervix using a multispectral digital colposcope [Benavides 2003] to identify cervical intraepithelial neoplasia (CIN) has also shown encouraging results. In a pilot study of 46 patients [Milbourne 2005], multispectral images could be matched to histopathology.

In some aspects, the systems, methods, etc., herein can provide improved delineation as compared to point tests (either spectral or tissue sample), can provide substantially instantaneous diagnosis because, if desired, there is no need to wait for laboratory analysis, report generation and return time. It can also save money in some instances by reducing or eliminating lab tests. It also may be possible to detect some lesions that can be missed by traditional white light examination, in the oral cavity for example, but can be clearly identifiable under fluorescence.

These and other aspects, features and embodiments are set forth within this application, including the following Detailed Description and attached drawings. Unless expressly stated otherwise or clear from the context, all embodiments, aspects, features, etc., can be mixed and matched, combined and permuted in any desired manner.

### BRIEF DESCRIPTION OF THE DRAWINGS

FIG. 1 depicts a schematic overview of exemplary elements of a system for a multipurpose tissue viewer as discussed herein. Viewing pieces are on the left, the handheld unit body with light source (either self-contained or fed via a light guide or other suitable optical system) are central, the distal endpieces for transmitting the light from the tissue, and (typically) back again, are on the right of the Figure.

FIGS. 2A and 2B depict top plan views of exemplary of a multipurpose head configured for intestinal examination comprising multiple light guides, air/fluid passages and tool passages.

### DETAILED DESCRIPTION

The following paragraphs provide definitions of some of the terms used herein. All terms used herein, including those specifically discussed below in this section, are used in accordance with their ordinary meanings unless the context or definition clearly indicates otherwise. Also unless expressly indicated otherwise, the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

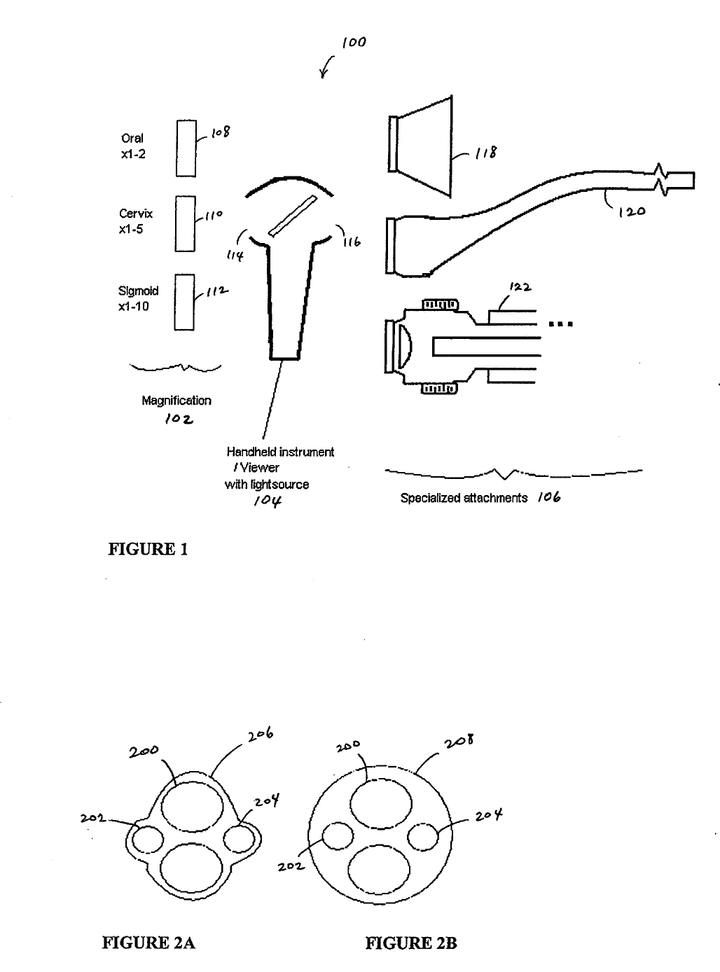

Turning first to a review of the Figures, FIG. 1 depicts a schematic overview of exemplary elements for a multipurpose tissue viewer 100 as discussed herein. Viewing pieces 102 are on the left, the handheld unit body 104 with light source (either self-contained or fed via a light guide or other suitable optical system) are central, and the distal endpieces 106 for transmitting the light from the tissue, and (typically) back again, are on the right of the Figure.

The viewing pieces 102 may include different viewing elements, each specialized to improve viewing of a particular tissue. For example, an oral viewing piece 108 may be suitable for viewing oral tissue and have a magnification range of approximately 1× to 2×, a cervix viewing piece 110 may be suitable for viewing cervical tissue and have a magnification range of approximately 1× to 5×, and a sigmoid viewing piece 112 may be suitable for viewing intestinal tissue and have a magnification range of approximately 1× to 10×.

Similarly, the attachments 106 may include different detachable instruments, each specialized to improve viewing of a particular tissue. For example, attachment 118 may be a skin cup for viewing skin tissue or a speculum attachment used for viewing body cavity tissue, attachment 120 may be a sigmoidoscope attachment used for viewing intestinal tissue, and attachment 122 may be an endoscope attachment used for viewing tissue inside the body and for enabling minimally invasive surgery.

The handheld unit 104 may include a first attachment point 114 and a second attachment point 116. Any of the viewing pieces 102 may be attached to the first attachment point 114, and any of the instruments 106 may be attached to the second attachment point 116.

FIGS. 2A and 2B depict top plan views of exemplary multipurpose heads configured for, e.g., intestinal or other internal examination. The heads include multiple light guides 200, air/fluid passages 202 and tool passages 204. The heads may also include a form-fitting extrusion 206 (FIG. 2A) or a circular extrusion 208 (FIG. 2B). If desired, the light guides 200 may be relatively large compared to the air/fluid passages 202 and the tool passages 204 in order to maximize illumination and viewing of the tissue.

Turning now to a more general discussion, the systems, devices, methods, etc., herein can, for example, use the handheld unit comprising a main body depicted in United States patent application U.S. patent publication no. 20050234526, or the rollerstand-based unit comprising a main body in U.S. patent publication no. 20060241347. Each of these units comprise a light source, typically a stand-alone light source such as internal or distally-located LEDs, an internal light source such as a light bulb or externally located light sources such as a proximally-located light bulb operably coupled by light guides to the main body; elements (typically light guides) for light delivery to a main unit; the main body(s) itself which comprises optical element(s) such as lenses and/or mirrors to direct the light to the target tissue and for managing concurrent light pathways (if so configured) for both illumination and directly viewing the tissue under test as well as other optical purposes if desired. The units, systems, etc., herein comprise several detachable instruments affixable to the handheld unit for the delivery of light to the tissue. The light delivery elements and detachable instruments can be specialized according to the type of tissue being interrogated. The systems, etc., can also comprise several detachable viewing eyepieces for the delivery of light to the user, which can be specialized according to the tissue being viewed. The systems, etc., can also comprise an optional disposable cover or window element for the protection of the patient and operator from spread of contagion.

Exemplary light sources. Light sources such as a metal halide light source—or other technology producing sufficient excitatory light, generally of blue light and/or longer wavelength UV light—is used to generate light. Examples of other efficient blue light sources include high pressure mercury and xenon arc lamps, light emitting diodes (LEDS), lasers, and electrodeless plasma lamps. The output of the light source is typically filtered to provide a narrower output spectrum, which improves specificity by limiting the molecules excited by the fluorescence. In one embodiment, the light is delivered via a light guide into the handheld unit. Delivery of the light from the light source to the handheld unit can be through use of a light guide such as a fiber optic light guide, liquid light guide or other. Another embodiment comprises LEDs located in the separate light source and then delivering it directly to the tissue. Alternatively, the light source can be built into the handheld unit so a light guide is not required. The light source can be AC powered or battery operated. If desired, a DLP such as a DMD (digital micromirror device) or MEMS can be used, for example for light spectra modification or light direction modification, for the provision of confocal capabilities or otherwise as desired.

In some embodiments, raw excitation light from the light source is conditioned by an excitation filter. The excitation filter can be placed in the handheld unit or it can be placed in the site-specific attachment for light delivery and collection or otherwise as desired. The excitation filters can be as close to the tissue site as possible in order to avoid the generation of unwanted autofluorescence in the optical elements downstream of the excitation filter.

Different organ and tissue sites can be advantageously visualized for the purpose of disease screening such as precancer screening when the correct band of excitation light is used. In other words, each tissue type has a band of excitation light which most clearly identifies precancerous and cancerous lesions. Some grades of lesions (for example moderate dysplasia) may be more easily identified with one excitation band of light than invasive cancer. This means that having the ability to easily change excitation filters is useful for a device which is used to screen for so many different diseases/cancers, particularly with cancer and other diseases progressing through several distinct stages over a long period of time.

The ability to change excitation filters can be implemented in the hand-held unit as a magazine or turret of several filters. This would also facilitate in changing between white-light and fluorescent imaging modes.

The ability to change excitation filters can be implemented in the site-specific attachment for light delivery and collection as a fixed filter. The filter can be change by attaching a different site-specific attachment.

Both white and blue (excitation) light can be used in diagnosis. For broad-spectrum light sources such as the metal halide, changing between light sources can be achieved by changing the output filters. For blue light a narrowband filter about the center wavelength can be used. For white light a broader bandwidth is used whereby all desired light in the visible spectrum is passed and unwanted light, such as ultraviolet and possibly infrared wavelengths, is stopped.

LEDs have narrower bandwidths and filtering may not be desired. As well, these are generally much cheaper and thereby multiple separate LEDs for blue light and white light (either white LEDs or a combination of red, green and blue LEDs) can be implemented in the same light source and turned on and off separately depending upon the light output desired.

Exemplary main body (usually handheld) units. The main body receives light from the light source, filters it as desired, delivers the light to the detachable light delivery instruments, and receives the light from the tissue and presents it to the user through the detachable eyepieces (the main body can also provide light from the tissue to electrical or other light detection systems, such as spectrometers or digital cameras, if desired).

Light routing through the main body unit can be through any desired approach/method, for example fiber bundles coupled to the input light source, free-space optics, total internal reflection waveguides and lightpipes, hollow-core waveguides, photonic-crystal fibers and others.

Filtering of the light such as for spectral and intensity content inside the main body unit can be done at the input to the main body unit, at interfaces between components as for butting fiber guides, between collimator lenses, or otherwise as desired.

Coupling of the light from the light-routing elements of the main body unit into the next modular attachment for projection onto the tissue site of interest can be accomplished for example by either butting two light-routing elements together or by coupling lens which can improve the optical efficiency. The coupling lenses can be a single lens in either one or the other of the two mating pieces or each mating piece (i.e., the main body unit and the attachment(s)) can have its own lenses. The lenses can be refractive lenses with spherical or aspherical surfaces or reflective lenses with for example, parabolic or elliptical surfaces. One embodiment would be to use a set of graded-index (GRIN) lenses bonded to the ends of the light guides.

The main body unit can be for example monocular, binocular with the same image is presented to both eyes or true stereo for 3-D imaging with a different image transmitted to each eye.

The main body unit is typically a light and fluid tight device. This improves detection and keeps contaminants (dust, blood, saliva, etc.) outside of the device.

Exemplary detachable instruments for light delivery. The detachable instruments for light delivery can be specialized according to the tissue that is being viewed and/or for other purposes such as the disease be investigated. Specific tissues under consideration for this application include oral, intestinal, cervical and skin. Others can be also possible such as Ear-Nose-Throat, (areas accessible by bronchoscopes, otoscopes, or nasoscopes), and the eye(s). (Unless expressly stated otherwise or clear from the context, all embodiments, aspects, features, etc., can be mixed and matched, combined and permuted in any desired manner.)

The detachable instruments can be reusable or disposable.

An optical purpose of the site-specific attachment is to project excitation light on the tissue of interest and collect fluorescent light. The detachable instruments can have a lens system to project light from the light-routing elements of the handheld unit onto the tissue at a particular working distance measured from the attachment. One embodiment uses an annular lens system (ring) of microlenses (e.g., refractive, reflective, graded index) which all focus the light to a common point on the tissue.

The detachable instruments also may include a lens or other optical device(s) to collect fluorescent light from the tissue and relay it to the eye piece. This lens and/or the eye piece can be used to magnify the image.

The working distance to the tissue can be determined by a lens or other optical device(s) in the distal end or tip of the attachment. Therefore, the detachable instruments can have a fixed focus (working distance) or a variable focus such as a fine focus such as by translating the distal attachment along the optical axis.

Exemplary Target Tissues/Structures:

Oral. One of the detachable oral instruments' purposes is to deliver light into the mouth. As such it is typically desired to shield ambient light. It can also be desirable to provide structural elements to move the cheek out of the field of view when desired. Exemplary adaptors to handle this are discussed in U.S. patent publication no. 20050234526 (this U.S. patent publication no. 20060241347 also show other adaptors suitable for various diseases/targets mentioned herein).

Intestinal. Intestinal detachable instruments provide light to the colon or other intestinal structures and receive light back from the illuminated tissue. The illumination light guide can be a liquid light guide. However, since the gastro-intestinal tract twists and bends, the light guide for viewing is preferably coherent such as a fiberguide in order that the image can be maintained.

Intestinal detachable instruments can be rigid, however to more comfortably navigate past the bend at the joining of the rectum and descending colon, it can also be flexible and can have a steering mechanism. The steering mechanism can comprise wires embedded in the device and anchored towards its distal tip, and can be tightened and released, tensioned and compressed, by knobs at the proximal end of the detachable intestinal head. Controls for the steering mechanism can be contained within this endpiece. The controls can also be on the handheld unit or otherwise as desired.

Depending on whether or not fluids (such as saline and/or drugs), air (e.g., suction) and tools (e.g., bioptomes, cutters) are desired to be delivered, lumens (hollow tubes inside the main body) within the intestinal detachable instruments, a version of the head can have these adjacent to the light guide(s). Cross-sections of form-fitting and circular devices are shown in FIG. 2. Circular extrusions are simpler. Form-fitting use less material, and may assist in traversing corners or to the grip of protective coverings, for example to cover certain holes and leave others open. FIG. 2 shows some devices particularly useful for intestinal examinations.

Cervical. Detachable instruments for viewing the cervix or other intra-vaginal structures allow light delivery and viewing inside the vaginal canal. A standard way of meeting these criteria for a gynecologist (or other health practitioner) is through use of a speculum: a hinged duckbill shaped tool that opens when squeezed. A speculum-shaped attachment can be used with the devices and methods herein. The speculum can be separate from or integral with the rest of the detachable instrument. Exemplary adaptors to handle this are also discussed in U.S. patent publication no. 20060241347, including adaptors useful for Pap smears.

Skin. Since skin is exposed, viewing skin fluorescence in a lit room will typically involve shielding the area of interest from extraneous light. Such barrier can have the shape of an inverted cup (such as a rounded pyramid or a cone) that is placed over the region of interest. This can attach to the handheld unit.

Detachable viewing eyepieces. A concern for the eyepiece(s) is magnification; the amount can vary from application to application (e.g., by tissue or disease). As noted above, the various embodiments discussed herein can be combined, replaced for each other, permuted, etc., as desired.

Adjustable magnification can be achieved for example by having multiple eyepieces which are easily attachable and each with its own magnification. Alternatively, an eye-piece attachment containing a magazine or turret of different magnification lenses can be employed. Also alternatively, the eyepiece can be continuously variable by manually twisting the eyepiece and thereby changing the relative location of optical elements within the system to provide variable magnification.

The eyepiece sections can be configured to contain and to allow easy change of the emission light filters, which can be, e.g., short pass, long pass, band, notch, polarization, etc., filters. The specific filters will depend on, e.g., the disease, organ and/or tissue type under investigation and more than one filter may be appropriate for the same disease or tissue type, etc. In one embodiment, a magazine or turret of several emission filters can be provided.

Not all tissues, such as oral tissue, usually need magnification for viewing. However, when desired, in one embodiment removable lens pieces with a range of magnifications between one and five times can be provided; such a range is useful for many other tissues, such as cervical and skin.

Disposables devices, e.g., useful for Cleanliness. The detachable instruments and other parts of the systems herein can be re-sterilizable or cleanable.

Disposable devices can be either the detachable instruments themselves or provided in combination with non-disposable detachable instruments (or in other aspects of the devices, systems, methods, etc., herein) used as a barrier to infection. Much like the detachable instruments and eyepieces, these can be specialized according to the given situations, such as the detachable instrument, the tissue, etc. they can be to be used with.

Barriers placed in the viewing and illumination paths should be optically clear, at least in the given wavelengths under consideration and preferably are tight to the surface of the detachable instruments, etc., and/or with a flat face to prevent optical distortions.

Oral disposable devices for certain detachable instruments including oral detachable light delivery pieces, are discussed in U.S. patent application Ser. No. 11/016,567, filed Dec. 16, 2004.

Intestinal disposable devices. Sigmoidal and other gastro-intestinal scopes become very dirty with use. As such a barrier similar to a condom can be convenient. Should there be no need for delivery of air, fluids or tools, the condom can be as simple as a bag pulled tight around the light guide.

However, if air, fluids and tools are desired to be delivered to the distal site, a lumen alongside the light guide configuration can be desired for their delivery. This complicates the barrier to contamination. As mentioned above in the detachable instruments section, the tubing can be shaped, so the condom in an appropriate position can be configured to that design, and can have holes to. The condom can have legs that ascend into the holes as well while leaving them open to the passage of the fluids, air and tools.

Cervical disposable devices. For viewing the cervix such as in the usual cervical examination, a speculum can be used to open up the vaginal canal. The speculum can be disposable.

Skin disposable devices include. The cup discussed above can be disposable.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “means” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion herein.

What is claimed is:

1. An autofluorescence light examination system comprising a plurality of modular parts including a) a main body configured to deliver light to a target tissue and containing at least one optical element configured so that a user can directly view the target tissue through the main body while the target tissue is illuminated by the light source, the main body further configured to accept at least two detachable instruments that are configured to deliver light to at least two different target tissues located at at least two different areas of the body and configured such that a user can directly view the target tissue through the main body while the target tissue is illuminated by the light source, b) the light source, and c) the at least two detachable instruments.

2. The system of claim 1 wherein the system further comprises at least 3 detachable instruments configured to deliver light to at least three different target tissues located at at least three different areas of the body.

3. The system of claim 1 wherein the system further comprises at least 4 detachable instruments configured to deliver light to at least four different target tissues located at at least four different areas of the body.

4. The system of any one of claims 1-3 wherein the system further comprises at least two detachable viewing eyepieces configured for the delivery of examination light to the user while the user can directly view the target tissue through the main body while the target tissue is illuminated by the light source.

5. The system of claim 4 wherein the at least two detachable viewing eyepieces are configured to deliver light from at least two different target tissues located at at least two different areas of the body.

6. The system of any one of claims 1-5 wherein the system further comprises a disposable window element sized to cover a distal end of the detachable instrument and to protect the patient and user from spread of contagion.

7. A method of autofluorescence light examination of a target tissue comprising using a system according to claim 1 to directly examine with autofluorescent light multiple different target tissues located at multiple different areas of a body without changing the main body.

8. The method of claim 7 wherein the method further comprises using at least 3 different detachable instruments and examining at least three different target tissues located at at least three different areas of the body.

9. The method of any one of claims 7-8 wherein the method further comprises not changing the light source.

10. The method of any one of claims 7-9 wherein the method further comprises changing between at least two detachable viewing eyepieces configured for the direct delivery of examination light to the user while the user directly views the target tissue through the main body while the target tissue is illuminated by the light source.

11. The method of any one of claims 7-10 wherein the method further comprises changing between at least two different disposable window elements sized to cover a distal end of the detachable instrument.

12. The method of any one of claims 7-11 wherein the method further comprises obtaining a digital or photographic image of the target tissues under examination.

13. The method of any one of claims 7-12 wherein the method further comprises obtaining at least one biopsy of at least one of the target tissues under examination.
